(19) United States
(12) Patent Application Publication
(10) Pub. No.: US 2013/0055619 A1
(43) Pub. Date: Mar. 7, 2013

(54) SCENT-DISPERSING SYSTEM AND METHOD FOR ATTRACTING WILD ANIMALS

(71) Applicant: Keith J. STEWART, Aliceville, AL (US)

(72) Inventor: Keith J. STEWART, Aliceville, AL (US)

(21) Appl. No.: 13/225,641

(22) Filed: Sep. 6, 2011

(51) Int. Cl. A01M 31/00; B05B 3/18; A61L 9/04

(52) U.S. Cl. 43/1; 239/34; 239/722

(57) ABSTRACT

Scent-dispersing systems include a pulley/line assembly and one or more scent-dispensing elements. The pulley/line assembly includes two or more pulleys that mount to trees or other fixed structures at positions above the ground and a laterally disposed line that is routed around the pulleys. The scent-dispensing elements couple to the lateral line and, upon the lateral line being pulled around the pulleys in one direction or the other, are extended out away from or retracted back toward a base one of the pulleys. Thus, by winding out or retracting the lateral line relative to the base pulley, the scent-dispensing elements can be moved out to desired elevated positions at certain distances away from the base pulley, all without having to walk across the ground out to those positions.

### TECHNICAL FIELD

The present invention relates generally to the use of scents for attracting animals in the wild, and particularly to scent-dispersing systems and methods for use by hunters and others wanting to avoid detection by and thereby gain proximity to wild game animals.

### BACKGROUND

For many years hunters have tried to fool the nose of their game-animal prey with cover scents to attract wild game and/or to mask human-related odors (e.g., human body odor, oil from weapons, synthetics/plastics from gear, etc.). For example, hunters often disperse attractant scents to try fooling their quarry into perceiving a breeding opportunity, a buck/intruder threat, or another situation by which the wild game animal is lured into proximity to the hunter, all with the hope of bagging the trophy of a lifetime. Such scents typically include attractants such as food or animal scents (e.g., buck/bull urine, doe/cow urine, estrous secretions, or tarsal and other gland secretions), and are typically dispersed by application of the scent to the hunter's boots, drag rags, scent canisters, etc. For example, it's common to apply scents to scent bags that are then fitted onto the hunter's boots. Then as the hunter walks to his hunting location, the scent is dispersed from the scent bag onto the ground, thereby laying a trail that his game will follow, right to his hunting location.

But using boot-mounted scent bags, as well as other known scent-dispersing systems, requires the hunter to disturb the hunting location (e.g., rustle up leaves and snap branches), make noise, and/or place human-related odor in the air and/or on the ground as the hunter walks to strategically place his attractant scent in the hunting area. This leaves human-related scent in the woods, even when the hunter is not present, which tends to keep the prey away. If a game animal visits the hunting site once and detects any type of unnatural odor or finds that the lure is not real, that animal will not normally return to that spot again for a second opportunity for the same scent.

Years of time and research, not to mention probably millions of dollars, have been spent in efforts to mask or eliminate human-related odor so a hunter can go undetected in the woods. But it's become generally accepted that human odor cannot be completely eliminated. And wild game animals survive by smell, sight, and hearing, with the most hunted big game in North America—the whitetail deer—having the keenest sense of smell. You may get lucky and fool him once, but twice is near impossible. So even when using known scent-masking systems for masking human-related odors, the hunter still leaves some human-related scent in the hunting area when using known scent-dispersing systems such as boot-mounted scent bags to disperse attractant scents.

Accordingly, it can be seen that needs exist for improved ways to disperse attractant scents in the wild to attract wild animals into closer proximity while avoiding detection by them. It is to the provision of solutions to these and other problems that the present invention is primarily directed.

### SUMMARY

Generally described, the present invention relates to systems and methods for dispersing attractant scents to attract wild animals into a desired proximity while avoiding detection by them. In typical embodiments, the scent-dispersing systems include a pulley/line assembly and one or more scent-dispensing elements. The pulley/line assembly includes two or more pulleys that mount to fixed structures (e.g., trees or ground stakes) at positions above the ground and a laterally disposed line (typically generally horizontal) that is routed around the pulleys. The scent-dispensing elements couple to the lateral line and, upon the lateral line being pulled around the pulleys in one direction or the other, are extended out away from or retracted back toward a base one of the pulleys. Thus, by winding out or retracting the lateral line relative to the base pulley, the scent-dispensing elements can be moved out to desired elevated positions at certain distances away from the base pulley, all without having to walk across the ground out to those positions. In this method of use, the invention can be used by hunters of wild game animals or by other persons wanting to gain proximity to wild animals (e.g., wildlife photographers and zoologists).

The specific techniques and structures employed to improve over the drawbacks of the prior systems and methods and accomplish the advantages described herein will become apparent from the following detailed description of example embodiments and the appended drawings and claims.

### BRIEF DESCRIPTION OF THE DRAWINGS

FIG. 1 is an elevation view of a scent-dispersing system according to a first example embodiment of the present invention, showing a pulley/line assembly and three scent-dispensing elements suspended from it.

FIG. 2 is an elevation view of a base portion of the scent-dispersing system of FIG. 1, showing a base pulley, a lateral line routed around it, and a line puller in position for use.

FIG. 3 is an elevation view of a remote portion of the scent-dispersing system of FIG. 1, showing a remote pulley and the first scent-dispensing element suspended from the lateral line in position for use.

FIG. 4 is an elevation view of an elevated portion of the scent-dispersing system of FIG. 1 in use, showing the first scent-dispensing element being suspended from the lateral line.

FIG. 5 shows the scent-dispersing system portion of FIG. 4, with the line puller being operated to advance the first scent-dispensing element away from the base pulley.

FIG. 6 shows the scent-dispersing system portion of FIG. 4, with the second first scent-dispensing element being suspended from the lateral line.

FIG. 7 shows the scent-dispersing system portion of FIG. 4, with the line puller being operated to advance the first and second scent-dispensing elements farther away from the base pulley.

FIG. 8 shows the scent-dispersing system portion of FIG. 4, with the last scent-dispensing element suspended from the lateral line and all of the scent-dispensing elements advanced into a desired elevated position for dispensing and dispersing the scent.

FIG. 9 is an elevation view of a scent-dispersing system according to a second example embodiment of the present invention, showing a pulley/line assembly and three scent-dispensing elements suspended from it, and including ground stakes for mounting and suspending the pulley/line assembly.

FIG. 10 is a plan view of a scent-dispersing system according to a third example embodiment of the present invention, showing a pulley/line assembly and three scent-dispensing elements suspended from it, with the pulley/line assembly adapted for mounting to three trees.

### DETAILED DESCRIPTION OF EXAMPLE EMBODIMENTS

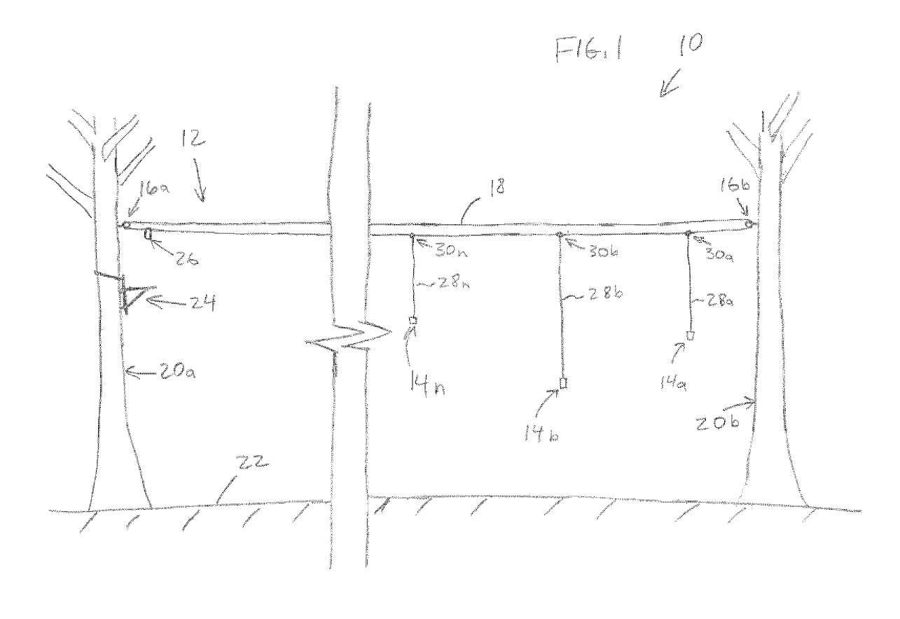

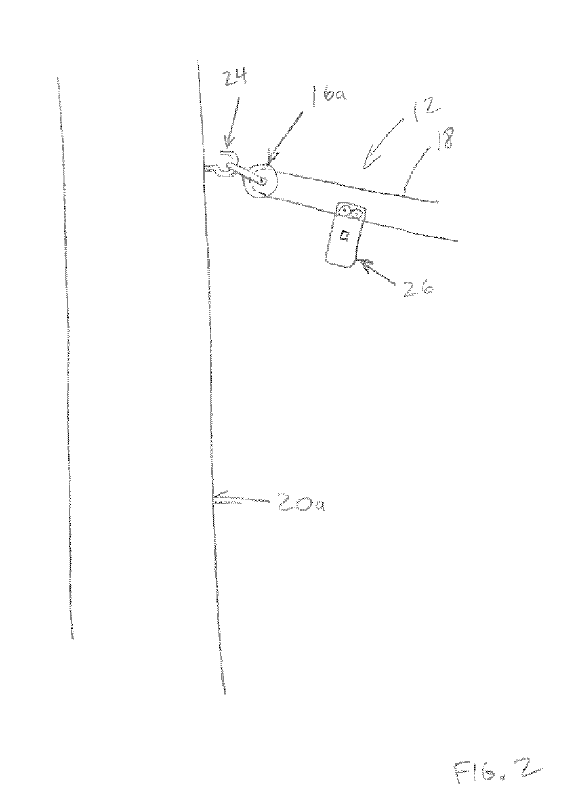

Example embodiments of the invention will now be described with reference to the drawing figures, in which like reference characters refer to like elements throughout. FIGS. 1-3 show a scent-dispersing system 10 according to a first example embodiment of the present invention. The system 10 includes a pulley/line assembly 12 and one or more scent-dispensing elements (collectively, the “scent-dispensing elements 14”). The pulley/line assembly 12 is operable to advance and retract the scent-dispensing elements 14 out and back in to and from various positions as may be desired during use.

The pulley/line assembly 12 includes at least two pulleys (collectively, the “pulleys 16”) and a lateral line 18 that is routed around the pulleys and advanced or retracted as the pulleys rotate in a first rotary direction or in a second opposite rotary direction. The pulleys 16 can be of a conventional type having a wheel that rotates on an axle shaft and that has two circumferential flanges defining a groove between them, all made of metal, plastic, nylon, carbon, or another suitable material. Alternatively, the pulleys can be provided by drums, rollers, sheaves, or other rotary mechanisms around which the lateral line can be run to change its direction.

The lateral line 18 can be of a conventional type of fishing line selected for having a light weight yet a high strength for good durability and also having a small diameter and low-visibility color for minimizing the likelihood of it being detected by wild animals. For example, clear or camouflaged monofilament or braided fishing line can be used for its low visibility characteristics. In a typical commercial embodiment, the fishing line can be of a type marketed under the STREN trademark by PURE FISHING, Inc. of Spirit Lake, Iowa. Alternatively, the lateral line 18 can be provided by a length of rope, string, cord, wire, cable, thread, belt, or any other thin elongated element that can be run back-and-forth around the pulleys, possibly even a chain. The lateral line 18 is routed around the pulleys 16 with its two ends connected (e.g., by a tied knot, crimp connector, splint connector, or other inline connector) to form a closed loop so that pulling the line in a first linear direction or an opposite second linear direction causes the looped line to rotate around the pulleys in the manner of a conventional clothes line.

The pulleys 16 mount to at least two fixed structures, typically trees in the wild (collectively, the “fixed structures 20” or the “trees 20”), at elevated positions above the ground 22. Alternatively, one or more of the fixed structures that the pulleys 16 mount to can include shrubs, bushes, a board or rope extending between two trees, a rigid vine hugging a tree, or any other natural or manmade structure that is fixed in place and to which the pulleys can be mounted in elevated positions. In some situations, one of the fixed structures that the pulleys 16 mount to can even be the ground itself.

The pulleys 16 can be mounted to the fixed structures 20 by conventional mounting elements 24. For example, the mounting elements 24 can be provided by J-hook (depicted) or eye-bolt screw-threaded connectors that screw into the trees 20, by straps or ropes that wrap around the trees, by brackets (e.g., L-shaped) that can be nailed or screwed to the trees, or by any other mounting elements that can couple to the trees (or other fixed structures) and the pulleys 16 for the intended use of the invention as described herein, whether made of metal, plastic, nylon, rubber, or another suitable material.

In the embodiment depicted in FIG. 1, the fixed structure where the hunter is positioned (e.g., in a tree stand 24) is referred to as the base tree 20a, and the pulley mounted to it is referred to as the base pulley 16a. The additional fixed structures and their pulleys (such as those depicted) are referred to as the remote tree 20b and the remote pulley 16b.

Typically, the base pulley 16a is mounted to the base tree 20a just above the hunter's tree stand 24, about 10 feet to about 25 feet above the ground. And the remote pulley 16b is typically mounted to the remote tree 16b at a comparable elevation, though it can be mounted lower or higher as may be desired.

The length of the installed lateral line 18 (the distance between the base and remote pulleys 16) varies by installation, and is typically in the range of a few hundred feet to a few thousand feet (the actual length of the line, uninstalled and laid out straight, is twice the distance between the base and remote pulleys). For installations with particularly long runs of the installed lateral line 18, one or more intermediate remote pulleys can be installed between the base and distal-end remote pulleys. The lateral line 18 is routed around the intermediate remote pulleys 16, and preferably secured from displacement from it, to prevent undesirable sag of the lateral line.

The pulley/line assembly 12 is operable to advance and retract the line 18, and thus the scent-dispensing elements 14, out and back in to various positions (between base and one or more remote locations) as may be desired during use. This can be done by manually pulling on the line 18 in a first linear direction or an opposite second linear direction to cause the closed-looped line to rotate around the pulleys. For example, a hunter in a tree stand 24 can reach out with his hands and grasp the lateral line 18 at a location adjacent the base pulley 16a and pull it to advance and retract the line 18 carrying the scent-dispensing elements 14. Referring to FIG. 1, the hunter could pull on an upper parallel portion of the looped line 18 to advance the scent-dispensing elements 14 out away from the hunter, or pull on a lower parallel portion of the looped line to retract the scent-dispensing elements back to the hunter.

To facilitate this advancement and retraction of the lateral line 18 carrying the scent-dispensing elements 14, a line puller 26 can be provided (as depicted). The line puller 26 can be a conventional device also known as a line stripper or line remover used for removing/unwinding fishing line from reels. Such devices include one or more rollers, an electric motor that drives the rollers, a power source (e.g., batteries) for powering the motor, and a control system for controlling the motor. The control system includes an on/off actuator (e.g., a button that turns on the motor upon depressing and turns it off upon releasing) and can also include other control features such as a directional control (for rotating the rollers in forward and reverse rotary directions). The motor can be a two-way motor with forward and reverse rotary directions, or a one-way motor with the line puller 26 easily removable from the lateral line 18, turned 180 degrees, and reattached to now pull the lateral line in the opposite direction. The body of the line puller 26 can include a spring-biased clip arm for latching the line puller onto the lateral line 18 for use and then removing the line puller from the line for removal from the site after use. In alternative embodiments without such a removal feature, the lateral line 18 is routed through a hole in the line puller when the line is installed, then the line puller is left there for future use. In a typical commercial embodiment, the line puller can be of a type (commercially available as is, or a modified version thereof) marketed under the BERKLEY trademark by PURE FISHING, Inc. of Spirit Lake, Iowa. In alternative embodiments, the line puller is provided by another type of line advancing/retracting device, such as other mechanical tools (e.g., hand cranks or ratchets, or even a common spinning or fishing reel) or other electro-mechanical devices (e.g., motorized cranks or ratchets).

The scent-dispensing elements 14 couple to the lateral line 18 and are carried by it as the line is advanced and retracted relative to the base pulley 16a. The scent-dispensing elements 14 can be of a type known in the art such as a scent-dispensing pad, wick, drip container, atomizer/sprayer, or another type of conventional scent dispenser. Such scent pads and wicks can be made of, for example, an absorbent wool or felt material. The depicted scent-dispensing elements 14 are scent pads of the type that include a strap for mounting onto the hunter's boots, though this strap can be eliminated for use in the present invention. In a typical commercial embodiment, the scent pads can be of a type marketed under the CODE BLUE trademark by EBSCO Industries, Inc. of Birmingham, Ala., with a size of about 4 to about 5 inches.

For use, the scent-dispensing elements 14 are loaded with a scent, such as an attractant and/or a masking scent, as is known in the art. The scent can be any type of commercially available scent such as for use for whitetail deer, mule deer, elk, and predators such as bobcat, coyote, bears, hogs, etc. These scents include food attractants, estrous, dominant buck, calming scent, pellet shavings from breeder pens, etc. In some embodiments, the scent-dispensing elements are pre-loaded with a scent. And in other embodiments, the scent-dispensing elements are not pre-loaded with a scent, and instead and one or more scents are packaged and provided with the scent-dispensing elements or provided independently for loading by the user.

The scent-dispensing elements 14 can be suspended from the lateral line 18 by drop lines 28 that are coupled to the lateral line. The drop lines 28 can be of the same type material and construction as the lateral line, though they need not be as strong and thick. Alternatively, the drop lines can be provided by straps, thread, belts, rigid hangers, or other elongated elements, whether made of plastic, metal, wood, or another suitable material.

In typical embodiments, a plurality of the scent-dispensing elements 14a-n are suspended by a plurality of the drop lines 28a-n (collectively, the “drop lines 28”), with one scent-dispensing element per drop line. In the embodiment depicted in FIG. 1, for example, three scent-dispensing elements 14a, 14b, and 14n are suspended by three drop lines 28a, 28b, and 28n. Alternatively, a single scent-dispensing element can be suspended by a single drop line, a plurality of scent-dispensing elements can be suspended by a single drop line, or a plurality of scent-dispensing element can be suspended by a single drop line.

The lengths of the drop lines 28 can be selected based on the height of the lateral line 18 above the ground 22 to thereby position the scent-dispensing elements at a desired elevated position above the ground. The desired elevated positions above the ground of the scent-dispensing elements 14 typically ranges from about 1 foot to about 3 feet. The elevational position of the scent-dispensing elements 14 can be selected based on wind currents, the type of scent used for each scent-dispensing element, and/or other factors that vary by scent and lateral location. As such, it is common, but not required, that at least two of the drop lines 28 have different lengths to position at least two of the scent-dispensing elements 14 at different elevational positions.

The drop lines 28 can be removably coupled to the lateral line 18 by removable connectors that permit the drop lines and their respective scent-dispensing elements 14 to be hung from the lateral line 18 for each use and removed after each use. In the depicted embodiment, for example, three removable connectors 30a-n (collectively, the “removable connectors 30”) are provided by spring-biased, pivot-arm connectors (e.g., alligator clips) that apply a gripping force to the lateral line 18 (to hold the drop lines in position along the lateral line) and that are fixedly attached to the top ends of the drop lines 28a-n. The removable connectors alternatively can be provided by male and female wire connectors that snap together, other clips, zip ties, wire crimps, straps, clamps, snaps, hooks, or other conventional mechanical connectors that are usable to removably couple (e.g., by clipping, tying, or snapping) one line to another, conceivably even clothes pins or paper clips, and can be made of for example plastic, metal, or wood. Typically, the scent-dispensing elements 14 are fixedly coupled to the bottom ends of the drop lines 28.

In some embodiments, the scent-dispensing elements are removably coupled (e.g., by removable connectors of the same or a similar type) to the bottom ends of the drop lines to provide more flexibility in usage of the system (the same drop line can be separately used with different scent-dispensing elements), whether or not the top ends of the drop lines are removably or fixedly coupled to the lateral line. In some other embodiments, the scent-dispensing elements, drop lines, and lateral line are all integrally formed or fixedly coupled together as a unit. And in some other embodiments, the scent-dispensing elements are coupled directly to the lateral line without including the drop lines.

Having described the structure of the first example embodiment of the invention, its method of installation and use will now be described as another aspect of the invention. To install the scent-dispersing system 10, two (or more) fixed structures are identified or installed in the hunting area in positions that will result in a desired elevated placement of the lateral line 18 so that the scent-dispensing elements 14 can be run out to desired elevated positions for attracting the animal being hunted. Thus, in the depicted embodiment, the base and remote trees 20a and 20b are picked out by the hunter. Then the pulleys 16 are mounted to the trees 20, a length of the lateral line 18 is routed around them, and the ends of the lateral line are connected together to form a closed loop. For example, the hunter can climb the remote tree 20b, mount the remote pulley 16b to it by a mounting element 24 such as a J-hook connector, and route the lateral line 18 around that pulley. The hunter can then descend the remote tree 16b, grasp the ends of the lateral line 18, and walk over to the base tree 16a. Next the hunter would climb the base tree 16a while maintaining hold/control of the ends of the lateral line 18, climb the base tree 20a, mount the base pulley 16a to it by a mounting element 24 such as a J-hook connector, route the lateral line around that pulley, and connect the ends of the lateral line together to form a closed loop. The scent-dispersing system 10 is now installed and ready for immediate or later use. If for later use, the hunter would then descent the tree and depart the hunting area.

The drop lines 28 (with or without the scent-dispensing elements 14) can be installed now or they can be installed later (e.g., on hunt day). For the purposes of describing herein the methods of installation and use of the scent-dispersing system 10, the drop lines 28 are installed at the time of using the system 10 on day of the hunt.

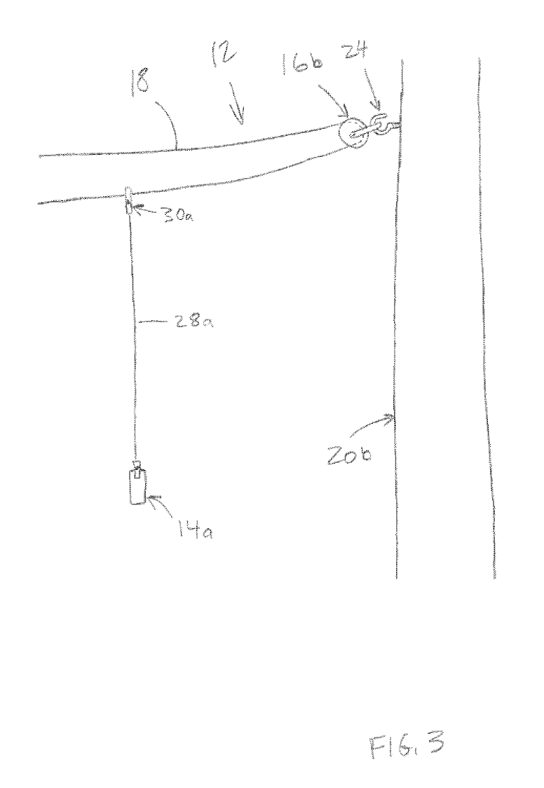

The use of the installed scent-dispersing system 10 will now be described with additional reference to FIGS. 4-8. First, the hunter climbs the base tree 20a carrying the scent-dispensing elements 14 and any needed drop lines 28 and/or scents (e.g., in a pouch). The first drop line 28a is selected for positioning the first scent-dispensing element 14a at the desired elevated position. If the first scent-dispensing element 14a has not already been loaded with a desired scent, that can be done now. Then the first scent-dispensing element 14a is coupled to and suspended from the lateral line 18, for example, by a removable coupling 30 (such as an alligator clip), as shown in FIG. 4. Next, the lateral line 18 is advanced, for example, using the line puller 26, to advance the first scent-dispensing element 14a away from the base pulley 16a (and thus the hunter), as shown in FIG. 5.

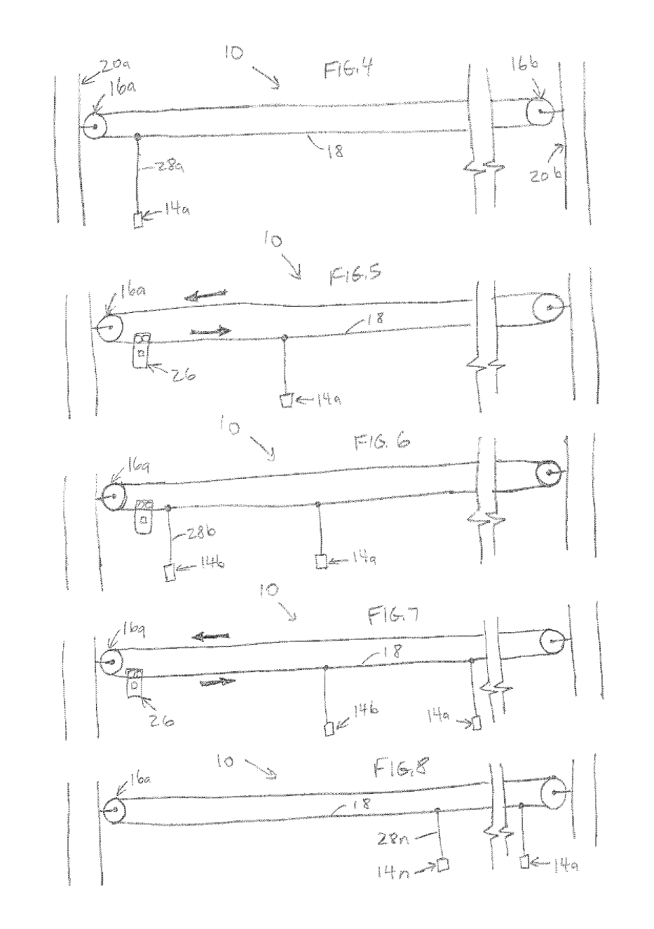

Then the second drop line 28b is selected for positioning the second scent-dispensing element 14b at the desired elevated position. Depending on the wind currents and thermals, scent used, animal hunted, ground terrain, etc., the second drop line 28b can be selected with a different length than the first drop line 28a. If the second scent-dispensing element 14b has not already been loaded with a desired scent, that can be done now. Then the second scent-dispensing element 14b is coupled to and suspended from the lateral line 18, for example, by a removable coupling 30 (such as an alligator clip), as shown in FIG. 6. Next, the lateral line 18 is further advanced, for example, using the line puller 26, to advance the second scent-dispensing element 14b (as well as the first scent-dispensing element 14a) away from the base pulley 16a (and thus the hunter), as shown in FIG. 7.

This process is repeated until the last drop line 28n and the last scent-dispensing element 14n have been coupled to and suspended from the lateral line 18 and run out to their desired elevated positions, as shown in FIG. 8. The line puller 26 can be left hanging from the lateral line 18 during the hunt, or it can be removed for now.

At the conclusion of the hunt, the scent-dispensing elements 14 and the drop lines 28 can be left in place, or they can be removed from the lateral line 18. To remove them, the above-described process is reversed. Thus, the lateral line 18 is retracted, for example, using the line puller 26, until the hunter can reach the last scent-dispensing element 14n and drop line 28n and remove them from the lateral line for storage (e.g., in a pouch). This process is repeated until the first scent-dispensing element 14a and drop line 28a have been removed. The hunter can then descend the base tree 20a and depart with the scent-dispensing elements 14 and drop lines 28, leaving behind the pulley/line system 12 intact for future use.

In this way, the hunter can place a desired scent in the air from an elevated position, so he does not need to walk across the ground 22 to get to the desired scent-dispensing location to disperse the scent. Also, the hunter does not need to later walk across that ground 22 to retrieve anything, as the pulley/line assembly 12 allows the scent-dispensing elements 14 to be retrieved from an elevated position. So the hunter never has to actually walk across the ground 22 along the trail where he wants to lure his prey (except for when installing the remote pulley at a much earlier time).

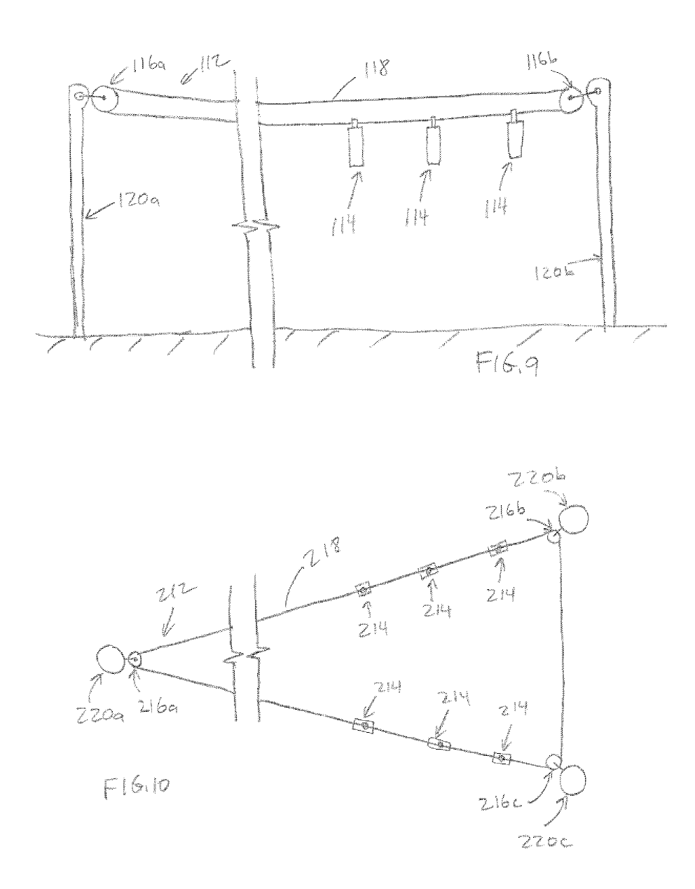

FIG. 9 shows a scent-dispersing system 110 according to a second example embodiment of the present invention. The system 110 is similar to that of the first example embodiment, in that it includes a pulley/line assembly 112 and one or more scent-dispensing elements 114. The pulley/line assembly 112 includes base and remote pulleys 116a and 116b (collectively, the “pulleys 116”) and a lateral line 118 routed around them, with the scent-dispensing elements 114 coupled to the lateral line. The pulley/line assembly 112 is operable to advance and retract the scent-dispensing elements 114 out and back in to various positions as may be desired during use.

However, in this embodiment the pulleys 116 are not mounted to trees. Instead, the system 110 includes one or more ground stakes (such as the depicted base stake 120a and remote stake 120b, collectively the “stakes 120”) that the pulleys 116 can be mounted to. The stakes 120 can have the form of conventional stakes, poles, or other elongated vertical members that can be implanted into the ground so that they extend vertically out of the ground. The stakes 120 can have a bottom end sufficiently pointed for driving into the ground. And the stakes 120 can have a top end that includes a loop, eyelet, or other mounting element to which the pulleys 116 can be easily mounted. The stakes 120 can be made of a high strength material (such as a metal or plastic) and have a height sufficient to place the scent-dispensing elements 114 at desired elevated positions. In typical embodiments, for example, the stakes 120 have length of about three feet to about five feet. Because the lateral line 118 is typically at a lower elevation in this embodiment relative to the first embodiment, the scent-dispensing elements 114 can be coupled directly to the lateral line without the need for drop lines.

This embodiment can be installed and used according to the same or a similar method as described above with respect to the first embodiment. The obvious difference is that the pulleys 116 would be mounted to the stakes 120 instead of to trees or other fixed structures. And of course the hunter would not need to climb the remote tree. In a related alternative embodiment, the hunter can use the remote stake 120b for mounting the remote pulley 116b and use the base tree for mounting the base pulley 116a.

FIG. 10 shows a scent-dispersing system 210 according to a third example embodiment of the present invention. The system 210 is similar to that of the first example embodiment, in that it includes a pulley/line assembly 212 and one or more scent-dispensing elements 214. The pulley/line assembly 212 includes base and remote pulleys 216a and 216b and a lateral line 218 routed around them, with the scent-dispensing elements 214 coupled to the lateral line. The pulley/line assembly 212 is operable to advance and retract the scent-dispensing elements 214 out and back in to various positions as may be desired during use.

However, in this embodiment the pulley/line assembly 212 includes three pulleys: the base pulley 216a and two remote pulleys 216b and 216n (collectively, the “pulleys 216”). Thus, when viewed from the top, the lateral line 218 forms a triangle. So two sets of the scent dispensing elements 214 can be placed, one on each of the lengths of the lateral line 218 adjacent the base pulley 216a. In this way, the hunter has more flexibility in placing scents in the air in staggered positions to better disperse the scents.

This embodiment can be installed and used according to the same or a similar method as described above with respect to the first embodiment. The obvious difference is that the pulleys 216 would be mounted to the three trees (or other fixed structures) 120 instead of just two. And the lateral line 218 would be advanced in a first direction when attaching the scent-dispensing elements 216, and in fact it would be advanced past the desired distal-end position of a first set of the scent-dispensing elements. Then a second set of the scent-dispensing elements 216 would be coupled to the lateral line 218. And then the lateral line 218 would then be retracted in an opposite second direction until the first and second sets of scent-dispensing elements 216 are in their desired end positions.

In an alternative embodiment, instead of a clothesline-like pulley/line assembly, there is provided a single line run between two mounting elements such as eyebolts that are attached to two fixed structures such as trees. A tram with a motor-driven roller runs along the line and scent-dispensing elements are coupled to the tram for example by drops lines. The tram includes an RF transceiver for control by a remote control unit that also includes an RF transceiver. With this embodiment, the hunter can control the location of the scent-dispensing elements from any desired location without the RF range of the components.

In other alternative embodiments, there are provided kits that include all of the components of the scent-dispersing systems including those described herein. Thus, the kits can include pulleys, mounting elements, ground stakes, lateral line, removable couplings, drop lines, scent-dispensing elements, and scents. In typical embodiments, the lengths of the drop lines included in the kit are about 12 inches to about 24 inches, and the length of the lateral line included in the kit is about 25 yards to about 500 yards (on one reel or multiple reels). In addition, the various components of the scent-dispersing systems can be painted, dipped, and wrapped (with adhesive sheets) with a camouflage pattern.

Accordingly, by using the scent-dispersing system, a hunter can prepare for any hunt with one prior visit to the hunting location. Most deer hunters prepare food plots in the early spring for the fall deer season. They return a few months prior to the opening of the season to scout the hunting location to find the ambush site that gives them the best chance of success (e.g., based on trails, scrapes, rubs, etc.). At this time, they choose a stand location, then hang a tree stand over or around heavily used animal trails (e.g., between bedding and feeding locations). They will saw/cut trees, vines, limbs, etc., to clear shooting lanes at that time so that later they can approach and enter their stand quietly. If they use an attractant scent, the hunter would normally walk into the area in which he made all these preparations to hang the scent from a limb, spray it on the ground, or pour it out of a bag onto the ground. But by so doing, the hunter has alerted animals in the area of his presence with sound and human odor. Any trophy hunter will say the best chance of bagging that trophy is your first hunt in the area. It is the element of surprise that keep taxidermist in business. If you the hunter does his homework and makes sure all conditions are right, this can be his best chance.

The present invention is a ground-breaking natural scent-dispersing system that can dramatically increase the hunter's chance of success. The pulley/line system would be placed into position when the hunter is on-site trimming limbs and branches and hanging his stand. On the day the hunter chooses to hunt this particular stand location, once in the stand all the hunter has to do is snap the scent-dispensing elements (e.g., already treated with a scent of choice) onto the lateral line (e.g., using the drop lines) and press the button on the line puller, and he can place the scent at whatever distance and height/level he chooses, both silently and without contaminating the hunting area by walking all around trying to place scent out in the area on foot. The drop lines can have different lengths to position the scent-dispensing elements at different elevated positions to catch different wind currents and thermals.

Also, anytime during the hunt, the hunter can reposition the scent-dispensing elements by pressing the button on the line puller to virtually silently move the scent to another location without ever putting his feet on the ground. As the wind may change, the hunter may see deer that are not in direct line with the wind or thermal, so he can move the scent to them virtually silently and with little or nor noticeable motion.

The scent-dispersing system of the present invention works in the manner of a trolley. The hunter merely climbs into his stand, removes the scent-dispensing elements from a bag, snaps them on the lateral line, and actuates the line puller, and the lateral line goes out to the distance desired. For example, if the hunter is in a small, tight, timber area, the lateral line may be set up to run out only about 75 yards. Or the hunter could be hunting a food plot and run the distance out a few hundred yards.

As the hunt comes to an end, the hunter can reel the lateral line back in, unsnap the drop lines and scent-dispensing elements, put them back in to a ZIPLOC bag (to keep the scent fresh), and leave the woods the same as when the hunter entered—undisturbed. The hunter leaves only with the scent-dispensing elements (and the drop lines if desired), and everything else stays in place ready for when the hunter returns for another hunt. Also, the scent-dispersing system is easily used on the ground for predator hunting just by simply attaching the pulley/line assembly to ground stakes.

It is to be understood that this invention is not limited to the specific devices, methods, conditions, or parameters of the example embodiments described and/or shown herein, and that the terminology used herein is for the purpose of describing particular embodiments by way of example only. Thus, the terminology is intended to be broadly construed and is not intended to be unnecessarily limiting of the claimed invention. For example, as used in the specification including the appended claims, the singular forms “a,” “an,” and “the” include the plural, the term “or” means “and/or,” and reference to a particular numerical value includes at least that particular value, unless the context clearly dictates otherwise. In addition, any methods described herein are not intended to be limited to the sequence of steps described but can be carried out in other sequences, unless expressly stated otherwise herein.

While the claimed invention has been shown and described in example forms, it will be apparent to those skilled in the art that many modifications, additions, and deletions can be made therein without departing from the spirit and scope of the invention as defined by the following claims.

What is claimed is:

1. A scent-dispersing system for enabling a user to gain proximity to an animal and for use with base and remote fixed structures, the scent-dispersing system comprising:
a pulley/line assembly including a base pulley, at least one remote pulley, and a lateral line that is routed around the base and remote pulleys to form a closed loop, wherein the base and remote pulleys mount to the base and remote fixed structures, respectively, at elevated positions above ground, and wherein pulling the lateral line in a first linear direction or an opposite second linear direction causes the looped lateral line to rotate around the pulleys in a first or opposite second rotary direction, respectively, to advance or retract the lateral line relative to the base pulley; and
at least one scent-dispensing element coupled to and carried by the lateral line as the lateral line and scent-dispensing element are advanced away from and retracted back toward the base pulley between base and advanced locations, respectively, wherein in the advanced position the scent-dispensing element is elevated above the ground, remote from the base pulley, and dispenses scent for sensing by the animal without the user traversing the ground to dispense the scent.

2. The scent-dispersing system of claim 1, wherein the fixed structures are provided by trees.

3. The scent-dispersing system of claim 1, wherein the pulleys are mounted to the fixed structures by mounting elements.

4. The scent-dispersing system of claim 1, wherein the mounting elements are J-hook or eye-bolt screw-threaded connectors.

5. The scent-dispersing system of claim 1, wherein the lateral line is provided by fishing line.

6. The scent-dispersing system of claim 1, wherein the scent-dispensing element is provided by a scent-dispensing pad, wick, drip container, or atomizer/sprayer.

7. The scent-dispersing system of claim 1, further comprising at least one drop line coupled to the lateral line and to the at least one scent-dispensing element to suspend the scent-dispensing element between the lateral line and the ground.

8. The scent-dispersing system of claim 7, wherein the at least one scent-dispensing element comprises a plurality of scent-dispensing elements, the at least one drop line comprises a plurality of drop lines, and at least two of the drop lines have different lengths.

9. The scent-dispersing system of claim 1, further comprising a line puller adapted to advance and retract the lateral line.

10. The scent-dispersing system of claim 9, wherein the line puller is provided by a line stripper or line remover for removing/unwinding fishing line from reels.

11. A scent-dispersing system for enabling a user to gain proximity to an animal and for use with base and remote trees, the scent-dispersing system comprising:
a pulley/line assembly including a base pulley, at least one remote pulley, and a lateral line that is routed around the base and remote pulleys to form a closed loop, wherein the base and remote pulleys mount to the base and remote trees, respectively, by mounting elements at elevated positions above ground, and wherein pulling the lateral line in a first linear direction or an opposite second linear direction causes the looped lateral line to rotate around the pulleys in a first or opposite second rotary direction, respectively, to advance or retract the lateral line relative to the base pulley;
at least one scent-dispensing element coupled to and carried by the lateral line as the lateral line and scent-dispensing element are advanced away from and retracted back toward the base pulley between base and advanced locations, respectively, wherein the scent-dispensing element is coupled to at least one drop line that is coupled to and suspended from the lateral line, and wherein in the advanced position the scent-dispensing element is elevated above the ground, remote from the base pulley, and dispenses scent for sensing by the animal without the user traversing the ground to dispense the scent; and
a line puller adapted to advance and retract the lateral line.

12. The scent-dispersing system of claim 11, wherein the mounting elements are J-hook or eye-bolt screw-threaded connectors, the lateral line is provided by fishing line, the scent-dispensing element is provided by a scent-dispensing pad, wick, drip container, or atomizer/sprayer, and the line puller is provided by a line stripper or line remover for removing/unwinding fishing line from reels.

13. The scent-dispersing system of claim 11, wherein the at least one scent-dispensing element comprises a plurality of scent-dispensing elements, the at least one drop line comprises a plurality of drop lines, and at least two of the drop lines have different lengths.

14. A method of dispersing a scent in an area to enable a user to gain proximity to an animal, comprising the steps of:
identifying or installing a base and at least one remote fixed structure;
mounting a base pulley and a remote pulley to the base and remote fixed structures, respectively, at elevated positions above ground;
routing a lateral line around the base pulley and remote pulleys to form a closed loop;
coupling at least one scent-dispensing element to the lateral line in a base position adjacent the base pulley;
pulling the lateral line in a first linear direction to cause the looped lateral line to rotate around the pulleys in a first rotary direction to advance the scent-dispensing element away from the base pulley from the base position to an advanced position, wherein in the advanced position the scent-dispensing element is elevated above the ground, remote from the base pulley, and dispenses scent for sensing by the animal without the user traversing the ground to dispense the scent.

15. The scent-dispersing system of claim 14, further comprising the step of pulling the lateral line in an opposite second linear direction to cause the looped lateral line to rotate around the pulleys in an opposite second rotary direction to retract the scent-dispensing element toward the base pulley from the advanced position to the base position.

16. The scent-dispersing system of claim 14, wherein the scent-dispensing element is coupled to a drop line, and wherein the step of coupling at least one scent-dispensing element to the lateral line comprises coupling the drop line to the lateral line.

17. The scent-dispersing system of claim 16, wherein the step of coupling the drop line to the lateral line comprises coupling a plurality of drop lines to the lateral line, wherein the drop lines are coupled to a plurality of scent-dispensing elements, and wherein at least two of the drop lines have different lengths.

18. The scent-dispersing system of claim 14, further comprising the step of selecting a length of the drop line based on a desired elevation above the ground of the scent-dispensing elements in the advance position.

19. The scent-dispersing system of claim 14, further comprising the step of removing the scent-dispensing elements from the lateral line.

20. The scent-dispersing system of claim 14, wherein the step of pulling the lateral line comprises engaging a line puller onto the lateral line and actuating the line puller to advance and retract the lateral line.
